(19) United States
(12) Patent Application Publication
(10) Pub. No.: US 2026/0171235 A1
(43) Pub. Date: Jun. 18, 2026

(54) Systems And Methods For Implementing Adjunct Technology To Facilitate A Surgical Procedure

(71) Applicant: Stryker Corporation, Portage, MI (US)

(72) Inventors: Ross Nave, Kalamazoo, MI (US); Slaven Sutalo, Byron City, MI (US); Martin Griffin, Orange, CA (US)

(73) Assignee: Stryker Corporation, Portage, MI (US)

(21) Appl. No.: 19/128,802

(22) PCT Filed: Nov. 13, 2023
(86) PCT No.: PCT/US2023/037179

§ 371 Date: May 9, 2025

Related U.S. Application Data
(60) Provisional application No. 63/424,557, filed on Nov. 11, 2022.

(51) Int. Cl. G16H 40/63; G16H 20/40; G16H 70/20

(52) U.S. Cl. CPC G16H 40/63; G16H 20/40; G16H 70/20

(57) ABSTRACT

A surgical management system and methods thereof to provide safer, accurate, intuitive, and efficient surgical workflows. The system may include a display interface, and a primary display. The display interface may receive manual count in and manual count out of surgical articles, and the data reader may facilitate electronic count in and electronic count out of the surgical articles. A visual indication is provided on the primary display if the manual count does not match the electronic count. The manual and electronic counts may be applied to multiple portions of the procedure, and to varying types of surgical articles. The system may prevent use of the data reader until pre-procedure protocols have been satisfied. The system may facilitate real-time updating of a fire risk assessment, initiating of a surgical drape timer, and determining of required surgical instrumentation following an indication of a revised surgical procedure.

### CROSS-REFERENCE TO RELATED APPLICATIONS

The subject patent application claims priority to and all the benefits of U.S. Provisional Patent Application No. 63/424,557, filed on Nov. 11, 2022, the entire contents of which are hereby incorporated by reference.

### BACKGROUND

Prior to and during a surgical procedure, it is imperative that clinical staff accurately perform all procedure protocols, for example, verifying patient identification and executing item counts. In particular, the clinical staff needs to ensure an accurate inventory of surgical sponges that are “counted in” to be used in the surgical field, and those “counted out” before conclusion of the surgical procedure to avoid a surgical sponge being inadvertently retained inside a patient. Often, the procedure protocols are performed manually, and outputs recorded on a whiteboard that is located in the operating room. For example, the clinical staff may count the surgical sponges by hand, which is prone to human error, and write the manual count on the whiteboard, which is prone to illegibility. More recently, surgical sponges have been tagged with radiopaque markers, barcodes, and/or wireless transponders, such as radiofrequency identification (RFID) tags, to facilitate electronically counting the surgical sponges wirelessly with a data reader. One exemplary manner by which this is implemented is disclosed in commonly-owned United States Patent Publication No. 2022/0246288, published Aug. 4, 2022, the entire contents being hereby incorporated by reference. Such adjunct technology addresses certain shortcomings with manual counting, yet it would be desirable to further integrate the technology into the various procedural protocols of the surgical procedure.

### SUMMARY

The present disclosure is directed to surgical management systems and methods thereof to provide safer, accurate, intuitive, and efficient surgical workflows. Many examples are disclosed in the context of counting surgical sponges, but may be extended to other instruments and aspects. The system may centralize relevant information on a primary display to allow surgical personnel to quickly ascertain the safety checks (e.g., pre- and/or intra-procedure protocols) that have or have not been performed, reconcile of the count of the surgical sponges through manual counting and electronic counting, maintain an updated fire risk assessment, and the like. The system may include a display interface to receive the user inputs. The display interface may be separate from the primary display. Alternatively, the display interface and the primary display may be integrated.

In many instances, the system may disable certain features until the requisite pre- or intra-procedure protocols have been satisfied. In the context article counting, the system intuitively provides for tracking counts based on type of article (e.g., sponges, needles, etc.), personnel handling the count, sub-procedures performed, and the like. Lastly, the centralization of information—including information previously recorded manually-permits for improved data and reporting by administrators.

Therefore, according to a first aspect of the present disclosure, a method of facilitating a surgical procedure with a surgical management system is disclosed. A first user input may be received with a display interface. The first user input is a manual count in of surgical articles being counted in to the surgical procedure. For example, a medical staff member may input that one, five, or more sponges are being counted in to the surgical procedure. The manual count is displayed on the display, or updated and displayed on the display as appropriate. The data reader may detect identification tags associated with the surgical articles being counted in to the surgical procedure. The one or more processors receive the identification information from identification tag(s). The one or more processors determine an electronic count in of the surgical articles based on the identification information. The electronic count in is displayed on the primarily display, or updated and displayed on the display as appropriate. If the manual count of the surgical articles does not match the electronic count in of the surgical articles, a visual indication may be provided. The visual indication may be highlighting of the rows, fields, or tiles in which the counts are being displayed. An optional audible indication may be provided.

As desired or at the conclusion of the surgical procedure, it is appropriate to count out the surgical articles. The display interface may receive a second user input of a manual count out of one or more of the of the surgical articles being counted out of the surgical procedure. Those surgical articles were previously counted in to the surgical procedure. The one or more processors update the manual count of the surgical articles based on the manual count in and the manual count out, and the updated manual count is displayed on the primary display. For example, the user may have counted in five surgical sponges, after which three are manually counted out. The “5” may remain presented on the primary display, but struckthrough, followed by a “2” in a row associated with the same sponge type. The arrangement indicates that five surgical sponges of that sponge type were previously counted in, three surgical sponges were counted out, and two remain counted in. In other words, each entry of the manual count in and the manual count out, prior to an updated entry being provided to the display interface, may remain displayed on the primary display. The strikethrough (or other indicia such as color coding) may be used to indicate it as a previous entry with each successive counting instance.

The one or more processors receive the identification information based on wireless detection, with the data reader, of the identification tag from one or more of the surgical articles to be counted out of the surgical procedure. The one or more processors determine an electronic count out the surgical articles based on the identification information of the one or more articles to be counted out. The primary display may display the manual count, the electronic count in, the electronic count out. If the manual count does not match the electronic count out of the surgical articles, the visual indication may be displayed.

In certain implementations, the one or more processors may determine an electronic count of articles remaining as counted in as a difference between the electronic count in and the electronic count out. The electronic count of articles remaining as counted in may be displayed, and the visual indication may be provided if the manual count does not match the electronic count of articles remaining as counted in.

In certain implementations, the methods disclosed herein may be used to account for different staff managing the count, for example, during a shift change (i.e., a “hand off”). The first manual count may be inputted by a first medical staff member. During the hand off, it is indicated to the system that the second staff member is assuming the count. An identification input indicative of a change in staffing may be received on the display interface or via the data reader. For example, the second staff member may scan his or her badge with an optical scanner of the data reader, or use the RFID capabilities of the data reader. Thereafter, the second staff member may count in or count out the surgical articles. For example, the second may input to the display interface a second manual count in of additional surgical articles being counted in to the surgical procedure. The one or more processors may update the manual count of the surgical articles based on the first manual count in and the second manual count in. The updated manual count may be displayed, which includes displaying the first manual count in and the second manual count in. The first manual count in and the second manual count in may be displayed in manner as being inputted by different staff members. For example, the numerical values of the sponges being counted in may be colored differently, or shapes or other indicia may be used.

In certain implementations, the system may be utilized to associate each of multiple counts with different aspects or portions of the procedure, also referred to herein as “sub-procedures.” For example, an emergent need may arise based on information discovered during a first portion of the surgical procedure; and separate sponge counting may be indicated or required for the emergent need. Additionally or alternatively, a second surgeon may be taking over responsibility of the procedure, and he or she may desire a separate sponge count. An exemplary method includes associating the manual count may with a first portion of the surgical procedure on the display interface. Indicia associated with a second portion of the surgical procedure may be generated. This may include creating a tile, tab, or list. With the indicia associated with the second portion of the surgical procedure selected, subsequent of surgical articles being counted in or out—either manually or electronically—are associated and display with the second portion. The count associated with the first portion of the surgical procedure may not be visible if the indicia associated with the second portion is selected, and vice versa. Thus, the user may selectively toggle between display screens to view and modify the counts associated with each portion of the procedure.

According to a second aspect of the present disclosure, a method of facilitating a surgical procedure with a surgical management system includes preventing the counting of articles prior to verification and/or satisfaction of pre- or intra-procedural protocols. In one variant, the one or more processors prevent the counting in of surgical articles to the surgical procedure with procedure information in an unverified state. The primary display displays tiles of grouped content, wherein at least one of the tiles of grouped content is visually deemphasized (e.g., “greyed out”). Stored procedure information including patient information and/or clinical information is received from a database. The information may be displayed on the primary display. Prior to or thereafter, additional patient information is received based on wireless detection of a patient tag associated with a patient by the data reader. This may include optical scanning of patient-worn barcode with the data reader. The one or more processors determine the procedure information to be in a verified state based on the patient information matching the stored patient information, after which the tile of grouped content may be emphasized (e.g., “brightened”). Further, with the procedure information being in the verified state, the one or more processors may enable the data reader to initiate the counting in of the surgical articles to the surgical procedure. If the patient information does not match the stored patient information, an alert may be provided.

In another variant, the method includes preventing, with the one or more processors, counting in of surgical articles to the surgical procedure with procedure information in an unverified state. A pre-procedure protocol including a series of actions to be performed before commencement of invasive aspects of the surgical procedure is displayed on the primary display. For example, one of the series of actions includes confirming surgical personnel. Stored procedure information including patient information and clinical information is received from a database. The primary display displays the patient information and the clinical information. The display interface receives inputs configured to satisfy each of the series of actions of the pre-procedure protocol. The primary display may alter indicia corresponding to each of the series of actions as each of the inputs is determined to have been performed to satisfy a respective one of the series of actions. Once all of the series of actions of the pre-procedure protocol being satisfied, the procedure information is considered to be in a verified state based. The one or more processors enable the data reader to initiate the counting in of the surgical articles to the surgical procedure based on the procedure information being in the verified state.

According to a third aspect of the present disclosure, a method of facilitating a surgical procedure by assessing fire risk is provided. The method includes receiving, from a database, stored procedure information. The one or more processors may determine an initial fire risk assessment based on the procedure information. Additionally or alternatively, the user may provide inputs to the display interface as to the procedural characteristics of the surgical procedure. The procedural characteristics may include use of alcohol-based solution, a surgical site being above a xiphoid, patient receiving supplemental oxygen, and/or use of instruments that may provide an ignition source. The one or more processors receive identification information based on wireless detection, with the data reader, an identification tag from a surgical instrument to be used during the surgical procedure. The one or more processors determine an updated fire risk assessment based on the initial fire risk assessment and fire risk associated with the surgical instrument. For example, if the surgical instrument is electrocautery, laser source, or a fiberoptic light source, the updated fire risk assessment may be increased. The updated fire risk assessment is displayed on the primary display, and if, appropriate, visual indicia or alerts may be provided if the updated fire risk assessment is above a predetermined threshold. The method may include displaying, on the primary display, a remedial checklist if the updated fire risk assessment is above a predetermined threshold, and preventing visualization of other aspects the primary display until the remedial checklist has been satisfied.

According to a fourth aspect of the present disclosure, a method of facilitating a surgical procedure with a surgical management system may include ensuring draping is not prematurely placed following use of alcohol-based product, thereby potentially increasing risk of fire. The method includes receiving, at the one or more processors, an indication of use of an alcohol-based product for preparing a surgical site. The step may include detecting, with the data reader, an identification tag from packaging associated with the alcohol-based product. Additionally or alternatively, images may be captured with a camera. The images may include the surgical site and a medical staff member. Machine vision utilizing artificial intelligence may be leveraged to analyze the images to predict that the medical staff member is applying a product to the surgical site in a manner consistent with how one would use an alcohol-based product. Based on the indication, the one or more processors may initiate a drape timer of a predetermined value, and the timer may be displayed on the primary display.

According to a fifth aspect of the present disclosure a method of facilitating a surgical procedure with a surgical management system may include providing intraoperative guidance to instrumentation needed based on a revised surgical procedure. The revised surgical procedure may be necessitated based on aspects of the originally planned procedure. One example includes a liver biopsy being necessitated based on lesions detected during an appendectomy. Since certain instrumentation may be common to both the originally planned procedure and the revised surgical procedure, the methods may display, on the primary display, instruments required to satisfy the needed instrumentation in view of the current instrumentation. The method includes receiving an inventory of current instrumentation being used during the surgical procedure. A user may provide a user input to the display interface indicating that a revised surgical procedure is indicated. The one or more processors access a database to determine an inventory of needed instrumentation of the revised surgical procedure. The inventory of current instrumentation is compared with the inventory of needed instrumentation. The primary display displays the required instruments to avoid duplicity. In certain implementations, the required instruments may be automatically requesting through a hospital inventory system. Should at least one of the required instruments be unavailable, alternative instruments may be displayed. The alternative instruments may be based on hospital inventory, surgeon preference, and/or compiled references of surgeons from similar surgical procedures.

### BRIEF DESCRIPTION OF THE DRAWINGS

FIG. 1 is a perspective view of a surgical management system;

FIGS. 2-11 are display screens of a primary display and, in some instances, a display interface of the surgical management system;

FIG. 12 illustrates an exemplary surgical article for use with the methods of the present disclosure;

FIG. 13 is a flowchart of a method for verifying information or completing protocols prior to commencement of the surgical procedure.

FIG. 14 is a flowchart of a method to reconcile a manual count and an electronic count of surgical articles, in particular surgical sponges;

FIG. 15 is a flowchart of a method related to fire risk assessment;

FIG. 16 is a flowchart of a method to determine whether a required prerequisite step has been performed in order to enable a surgical process to proceed;

FIG. 17 is a schematic representation of electronic components of the surgical management system; and

FIG. 18 is a flowchart illustrating a method for initiating a sub-procedure during a primary surgical procedure and maintaining a separate sponge count during related to the sub-procedure.

### DETAILED DESCRIPTION

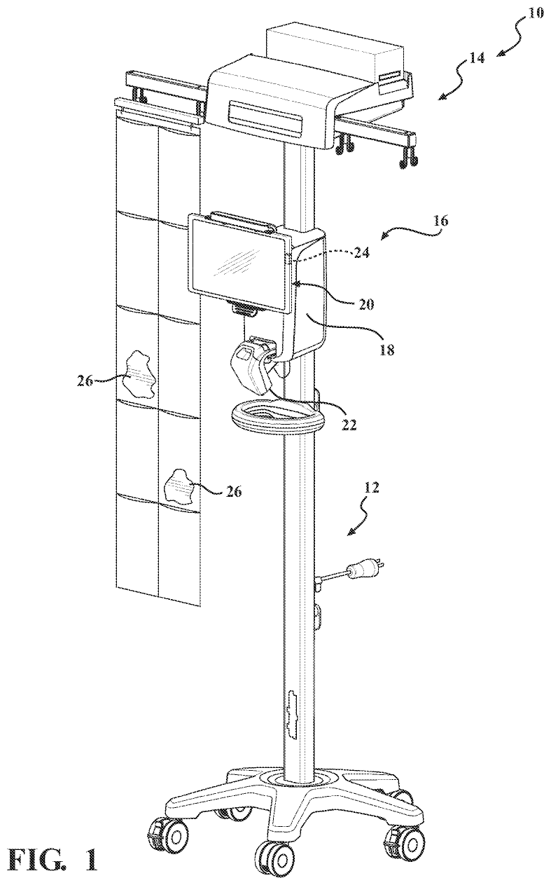

FIG. 1 shows a surgical sponge management system 10 including a stand 12, a dispenser assembly 14, and an electronics subsystem 16. The stand 12 supports the dispenser assembly 14 configured to store and ergonomically dispense sponge sorters and surgical draping. The electronics subsystem 16 includes a module base 18, a display interface 20, the data reader 22. The display interface 20 may be removably coupled to the module base 18. The display interface 20 may be a tablet displaying a graphical user interface (GUI). The tablet may include a processor 24, or alternatively the tablet may provide wireless connectivity with remote resources, such as a hospital network or internet server, for remote processing. The module base 18 may include a cradle configured to be removably coupled with the data reader 22. The illustrated implementation shows the cradle being a recess sized to receive and support the data reader 22 such that the data reader 22 is configured to be used as either a handheld device or while supported by the cradle, and seamlessly transition between the configurations. A primary display 21, such as a monitor separate from the tablet, may be positioned elsewhere within the operating theatre. The illustrated surgical sponge management system 10 may be exemplary, and other similar embodiments may be described for counting other surgical articles which may be dispensed in quantity during a surgical procedure.

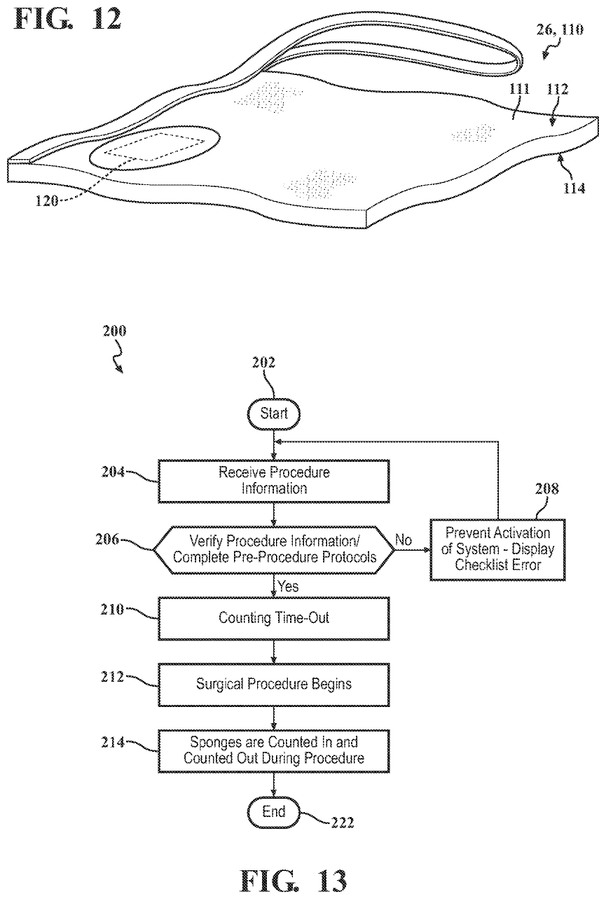

The data reader 22 may be an RFID scanner configured to detect RFID tags 120 associated with sponges 26, 110 (see FIG. 12). The RFID scanner includes the physical components and the operating software for generating interrogation signals and receiving responses to the interrogation signals. The physical components may include a signal-generating transmitter, and a signal receiver or transceiver, for example, as disclosed in commonly-owned International Publication No. WO2021/041795, published Mar. 4, 2021, and commonly-owned International Publication No. WO2021/097197, published May 20, 2021, each of which is hereby incorporated by reference in its entirety. Exemplary tags are disclosed in commonly-owned U.S. Pat. No. 8,181,860, published Oct. 2, 2008, and International Publication No. WO2017/112051, published Jun. 29, 2017, the disclosure of each being hereby incorporated by reference in its entirety. The surgical sponges 26, as used herein, may be any absorbent article, including but not limited to laparotomy pads, gauzes, towels, chux, and the like. Further, it should be understood that, as an alternative to surgical sponges, aspects of the present disclosure may be modified to be used with non-absorbent surgical articles including implants, clips, staples, or surgical instruments. For example, common-owned United States Publication No. 2019/0000589, published Jan. 3, 2019, hereby incorporated by reference, discloses implementations in which a scannable element and a human-readable element are disclosed on forceps and suture needles.

The surgical procedure—and the procedural protocols corresponding thereto—may include different phases, for example, preprocedural check-in, sign-in, time-out, and sign-out. Within those phases, the procedural protocols may indicate one or more steps, such as confirmation of patient identity, confirmation of surgical site, item counts, and the like. The system 10 of the present disclosure facilitates advancing through the procedural protocols and the phases of the surgical procedure in an efficient, intuitive, and error-preventing manner. Among other features to be described, the system 10 may require manually-performed steps to be confirmed with the adjunct technology, provide “lock outs” or triggers in the absence thereof, and provide visual indicators with successful or unsuccessful completion of various steps of the procedural protocols.

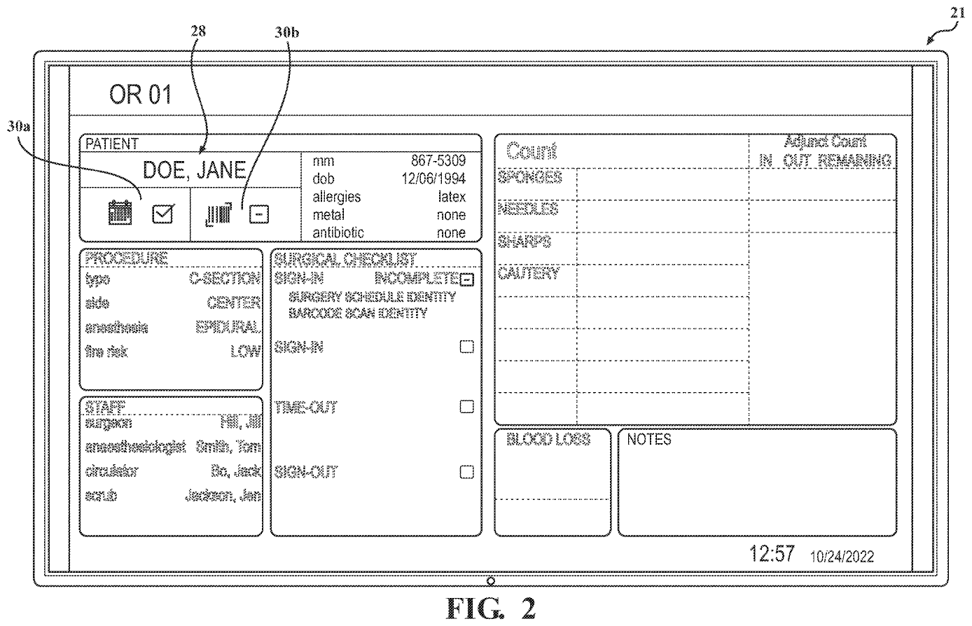

FIGS. 2-4 show an illustrative GUI of the primary display 21 for performing steps of a method for verifying patient identity. It is understood that the display outputs may alternatively or additionally be provided on the tablet. The GUI may be arranged in tiles of various sizes and shapes suitable to convey information in an intuitive manner. Of particular interest is a patient identification tile 28 in which identifying information of the patient is presented, including name, date of birth, allergies, and the like. Further, the patient identification tile 28 may include indicia 30a, 30b associated with patient verification. The processor 24 is configured to receive data indicative of a first patient verification input. The first patient verification input may be manually provided to the display interface 20. For example, the clinical staff may search for the patient by name, and/or by date and time of the surgical procedure. The processor 24 may be configured to communicate with electronic medical records (EMRs) of the hospital system to access and download the data. Once the patient is selected, the identifying information may be populated into the patient identification tile 28.

The processor 24 is configured to display the first indicia 30a indicative of the first patient verification input. FIG. 2 shows a checkmark adjacent a calendar icon to indicate to the clinical staff that the information presented was obtained through providing the patient name, date and time of the surgical procedure, and the like. However, since the adjunct technology has not yet been leveraged to verify the patient identity, the method may include “locking” or rendering inaccessible a remainder of the primary display 21. In other words, the otherwise selectable or displayable features of the primary display 21 may be greyed out and, if selected, perform no corresponding action. The second indicia 30b indicative of a second patient verification input may be color-coded or presented differently than the first indicia 30a to inform the clinical staff that the second patient verification input has not been provided to the system 10. As shown in FIG. 2, the second indicia 30b is presented as a negative sign, which is juxtaposed with the checkmark of the first indicia 30a. Further, while somewhat greyed out, another tile that presents the phases of the surgical procedure (i.e., check-in, sign-in, time-out, and sign-out), may be color-coded in a manner corresponding to the color coding of the second indicia 30b to inform the clinical staff the reason for the “lock out.”

The method includes receiving the second patient verification input. The second patient verification input may be provided using the adjunct technology. The data reader 22 may be configured to detect a tag storing the patient information. In one example, the patient may be wearing a wristband including an optical tag (e.g., barcode or quick-response code), and the data reader 22 includes an optical sensor configured to detect the optical tag. A prompt may be provided on the primary display 21 to instruct the clinical staff to remove the data reader 22 from the cradle to scan the optical tag of the wristband.

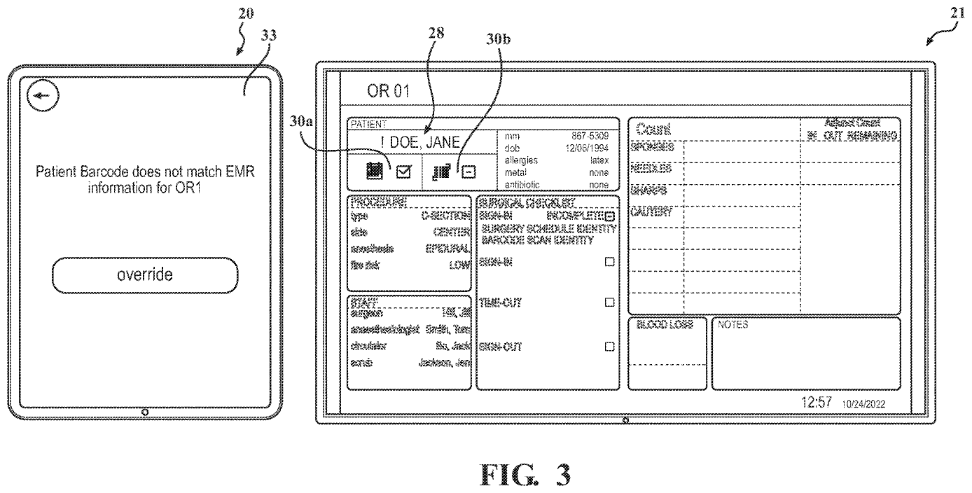

The processor 24 is configured to compare the second patient verification input with the first patient verification input. The second patient verification input not matching the first patient verification input may be indicative that the clinical staff manually entered the incorrect identifying information. FIG. 3 shows such an instance, wherein the second indicia 30b is updated to visually emphasize the inconsistency. The patient identification tile 28 is shown as an exclamation point, and the remainder of the primary display 21 remains greyed out or inaccessible. In certain implementations, a warning 33 may be populated on the display interface 20 to indicate that the second patient verification input does not match the first patient verification input. A selectable option may be provided to override the inconsistency, which would be stored and transmitted to the EMR to be associated with the surgical report.

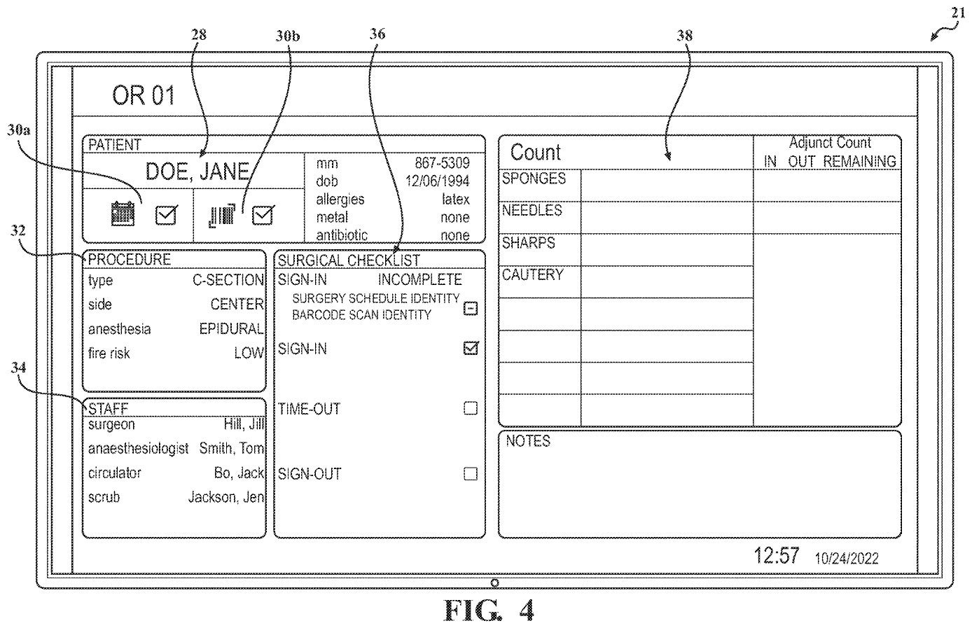

Should the second patient verification input match the first patient verification input, the processor 24 may be configured to alter the second indicia 30b to indicate successful patient verification. FIG. 4 shows the second indicia 30b being a checkmark to match the checkmark of the first indicia 30b. The system 10 may also provide audible feedback, such as a tone or noise to indicate the successful patient verification. The processor 24 may be further configured to enable visualization of more or the remainder of the primary display 21. FIG. 4 shows tiles 32, 34, 36, 38—other than the patient identification tile 28—brightened. Moreover, information has been populated into the procedure identification tile 32, and the staff identification tile 34. The surgical checklist tile 36 has been updated to indicate completion of the sign-in phase. Each of the first and second patient verification inputs are also listed beneath the sign-in phase. Other software or hardware functionality may become enabled once the processor 24 determines the patient identification has been confirmed using the adjunct technology.

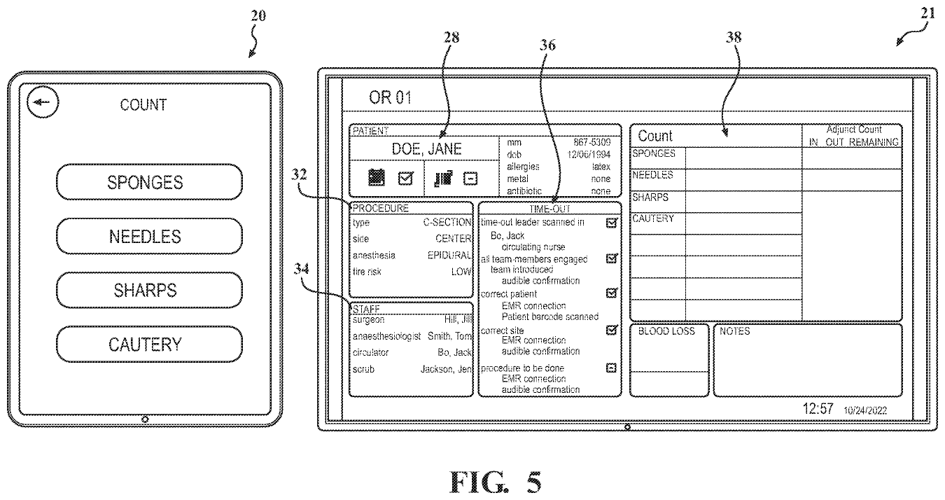

FIG. 5 shows another implementation of the surgical checklist tile 36 in which steps of a method of procedure verification are performed, also referred to herein as a “time-out.” The steps may include a leader of the clinical staff scanning in, the clinical staff introducing themselves, and confirming the surgical procedure and the surgical site. The method may include the processor 24 receiving a first personnel input indicative of the team leader scanning in. The team leader of the clinical staff may be wearing a badge including an optical tag, which may be scanned by the data reader 22. The method may include the processor 24 receiving a second personnel input indicative of the staff introductions. For the staff introductions as well as inputs corresponding to the confirmations of the surgical procedure and the surgical site, the display interface 20 may include a microphone (not identified) configured to detect sounds within the operating theatre. The sounds may be processed by the processor 24, using voice-recognition, artificial intelligence, or other means, to determine whether the staff has been introduced and whether the surgical procedure and the surgical site have been confirmed. The clinical staff may be trained to speak these items out loud. The processor 24 may compare the audible inputs against inputs received from the patient EMR (after the patient identity has been confirmed).

The surgical checklist tile 36 may be populated or may tabulate standard practices or hospital procedures which accompany or are prescribed for a given surgical procedure. In one example, four categories of checklists may be provided: a first, including a pre-procedure check-in process; a second, including a sign-in process; a third, including a time-out process; and a fourth, including a sign-out process. In one embodiment, the pre-procedure check-in process may include the patient or a patient representative actively confirming: identity, procedure and procedure site, consent, and optionally marking of the site. Further, the health care professional may confirm history and physical information, a pre-anesthesia assessment, a nursing assessment, diagnostic and radiology test results, blood products, and presence or history of special equipment, devices, or implants. Pre-procedure check-in may further include confirmation of beta-blocker medications, venous thromboembolism prophylaxis order, and normothermia measures.

In one embodiment, the check-in process may include reiterative confirmation of identity, procedure-to-be-performed, procedure site, and consent. The check-in process may further include confirmation of patient allergy information, pulse oximeter information, assessment of difficult airway or aspiration risk, risk of blood loss (for example, greater than 500 milliliters), confirmation of a number of blood units currently available, an anesthesia safety checklist complete indication, and confirmation that members of the team have discussed a care plan and addressed concerns.

In one embodiment, the time-out process may be operated before medical action is taken, i.e., pre-skin incision. A designated team member may be charged with suspending all activities during the time-out process. The time-out process may include the team members introducing themselves to the patient, reiterative confirmation of identity, procedure-to-be-performed, procedure site, and consent. The time-out process may further include confirming that the site is marked and visible, confirming a fire-risk assessment, confirming that relevant images are properly labeled and displayed, and confirming equipment status and identifying any equipment concerns. The surgeon may state critical or non-routine steps anticipated during the procedure, case duration, and anticipated blood loss. The anesthesia professional may confirm application of an antibiotic prophylaxis within one hour before the incision and may state any additional concerns. A scrub person and a registered nurse circulator may confirm that sterilization indicators are confirmed and may state any additional concerns. A health care professional such as a registered nurse may certify that the time-out process is complete.

In one embodiment, the sign-out process may be completed prior to the patient leaving the operating room. A health care professional such as a registered nurse may confirm completion of sponge, sharp, and instrument counts, may confirm that specimens taken are identified and labeled, and may identify equipment concerns to be addressed. The health care professional may further discuss wound classification and may record key concerns of every team member including concerns for recovery and management of the patient. The sign-out process may further include a debriefing with the team members including discussion of team performance, key events, and any permanent changes in a preference card.

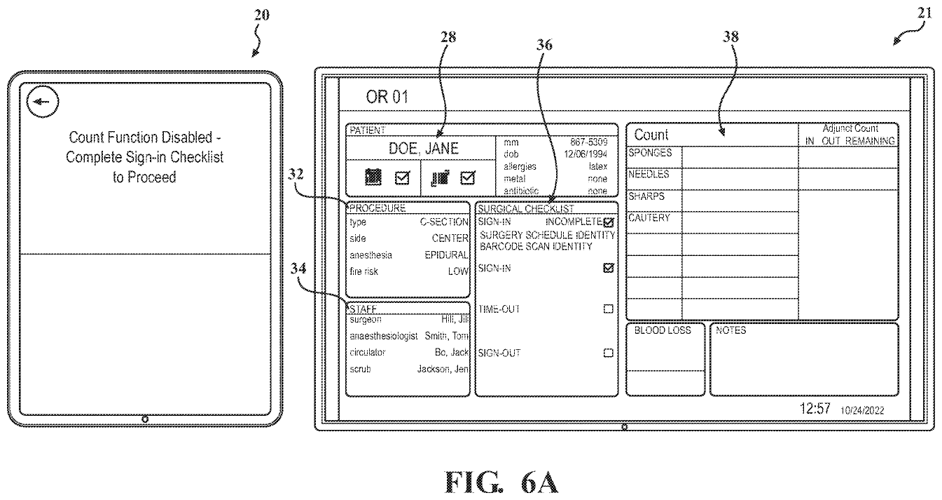

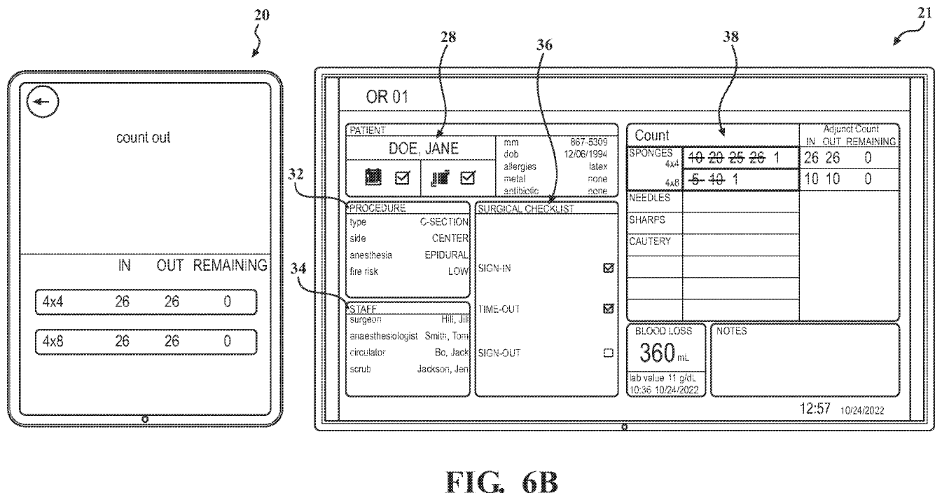

If the checklist process detailed within surgical checklist tile 36 is not completed successfully, the counting process described herein may be disabled. FIG. 6A schematically illustrates a failed checklist process described in the surgical checklist tile 36, wherein the sign-in step is described as being incomplete for failure of entry of one or more values. As a result, the display interface 20 provides a message instructing the health care professional to complete the incomplete sign-in checklist to advance towards enabling the count function disclosed herein.

Once the pre-requisite checklists detailed in the surgical checklist tile 36 are complete, the surgical aspects of the surgical procedure may commence. The surgical sponges 26 and/or other articles (needles, sharps, etc.) may be counted in to the surgical procedure. In particular, the processor 24 may be configured to identify the sponges 26 as being counted in or counted out based on detections of the RFID tags with the data reader 22. Referring now to FIG. 6B, the display interface 20 updates and provides the inventory identifying the types and quantities of the sponges 26 that have been counted in, and the types and quantities of sponges 26 that have been counted out. On the primary display 21, the article count tile 38 is configured to juxtapose the manual count with the electronic count (i.e., adjunct count from the adjunct technology). The processor 24 is configured to receive first article count inputs. The first article count inputs may be associated with the manual count and input by entering each instance one or more of the surgical sponges 26 are counted out of the surgical procedure. FIG. 5 shows each instance the first article count inputs to show the progression of the count out along with the current count out being highlighted. The article count tile 38 also includes the electronic count, which may be the same data reformatted from that being displayed on the display interface 20. In the illustrated example, the electronic count matches the manual count. A check mark or other indicia may be provided to further emphasize the same. In instances where the electronic count does not match the manual count, visual indicia or audible feedback may be provided. The visual indicia may be warning-style highlighting as shown, a prompt, or the like. Thus, while the manual counting and the electronic counting are performed separately, the system 10 of the present disclosure synergistically leverages the two to provide additional checks during the surgical procedure.

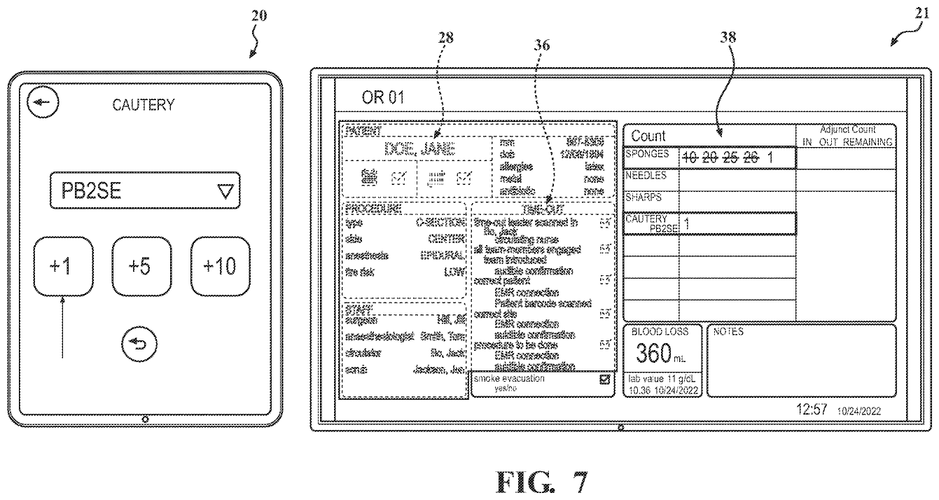

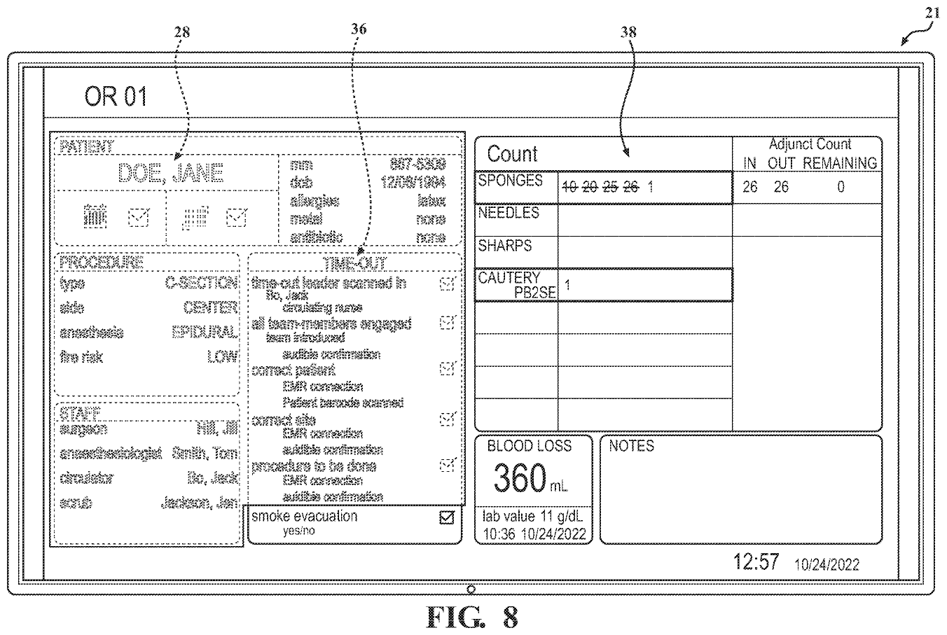

FIGS. 7 and 8 show the system 10 for use with cautery in which the display interface 20 cooperates with the primary display 21 to indicate that cautery is being used and at what level and/or duration. The processor 24 may receive a cautery input indicative of the clinical staff activating a cautery device (not shown), for example, on the display interface 20. The processor 24 may be configured to provide a corresponding output on the primary display 21. For example, the surgical checklist tile 36 of FIG. 7 shows indicia including a negative sign by “smoke evacuation,” indicating that smoke evacuation associated with the cautery has not been used and/or is not active. The clinical staff activates the cautery device at a plus one power level, after which FIG. 8 updates the surgical checklist tile 36 and the article count tile 38. The indicia of the surgical checklist tile 36 has been changed to a checkmark to indicate that smoke evacuation that may accompany the cautery has been used, and the article count tile 38 lists the cautery device as well as the power level. It is noted that FIGS. 7 and 8 show the patient identification tile 28 and-with the exception of the smoke evacuation option-the surgical checklist tile 36 as being greyed out, thereby focusing the user's attention on the smoke evacuation option. Alternatively, it is contemplated that the patient identification tile 28 and the surgical checklist tile 36 may be brightened (see FIGS. 6A and 6B).

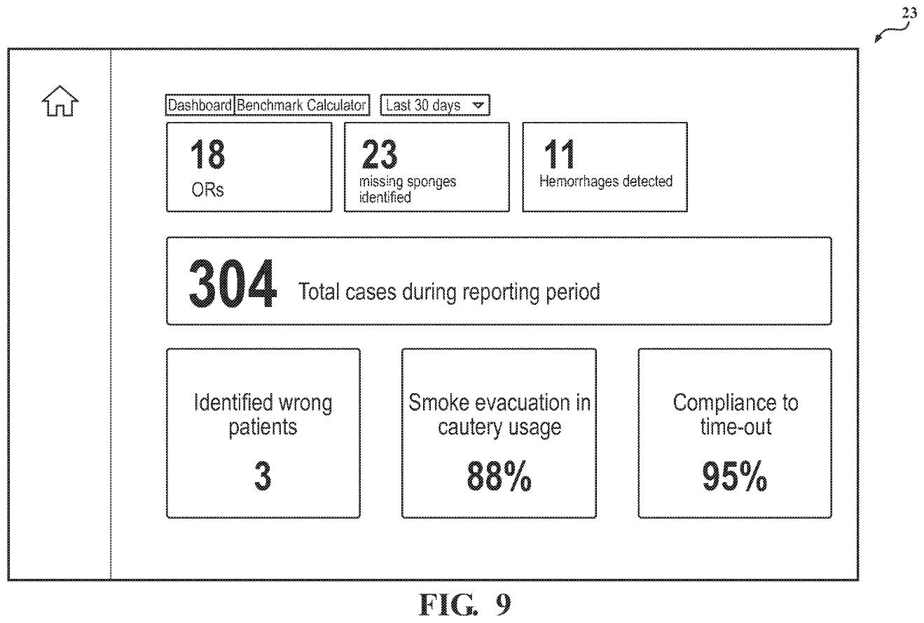

As mentioned, the processor 24 may communicate with the EMR to access patient data for verification and the like. It is contemplated that all of the aspects and data described herein may be collected and transmitted to the EMR, for example, to the cloud through means known to those skilled in the art. Moreover, the methods described herein may be leveraged to aggregate data across various patients, operating rooms, hospitals, clinical staff, and the like, to give a comprehensive analysis of compliance and best practices of the procedural protocols. FIG. 9 shows one such representation of an administrator display 23 in which a dashboard lists various metrics. Each of the tiles may be selectable to “drill down” into the underlying data supporting the metrics, for example, by viewing each surgical procedure in which the anomaly occurred.

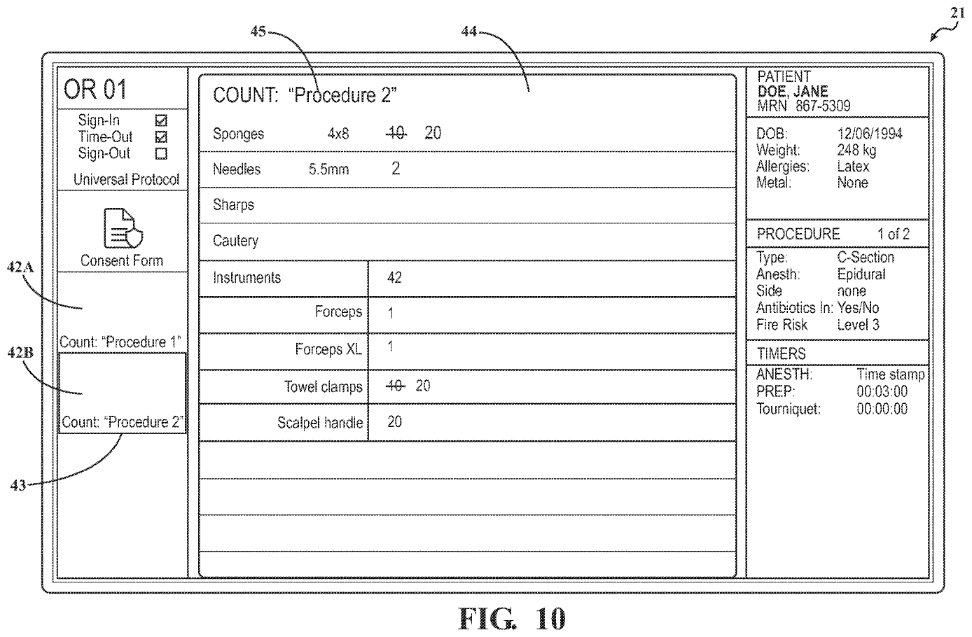

FIG. 10 shows the system 10 simultaneously maintaining separate counts for a first procedure and a second procedure. In one embodiment, the second procedure may be a sub-procedure to the first procedure. A sub-procedure may be planned, for example, with a surgeon intending to separately track one portion of the surgical procedure from a second portion. A sub-procedure may be emergent or unplanned, with an event or new information during the first or primary procedure necessitating implementation of the unplanned sub-procedure. The primary display 21 is illustrated displaying selectable outputs 42A, 42B directed to a Procedure 1 or a Procedure 2. A box 43 is illustrated indicating current selection of Procedure 2. Further, a banner 45 identifying current selection of Procedure 2 is displayed upon informational box 44 providing information about sponge and surgical article usages and counts related to procedure 2. A health care professional may provide an input to system 10 causing the primary display 21 to switch to displaying current information about Procedure 1. The primary display 21 may further be configured to show an overall count including information related to both Procedure 1 and Procedure 2 simultaneously.

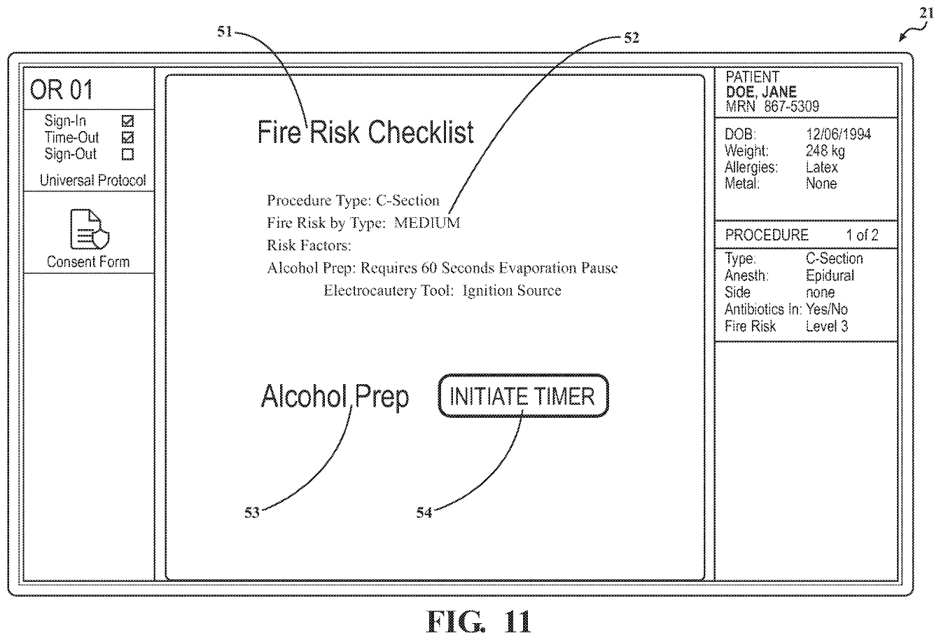

FIG. 11 shows the system 10 being configured for providing fire risk information to the viewer upon the primary display 21. The primary display is configured to display relevant information regarding the patient, already completed checklists, and an accessible copy of an executed consent form. The primary display 21 is further configured to display fire risk information in the form of a fire risk checklist, as identified by banner 51. A list 52 of information related to a fire risk associated with the current procedure is displayed. The list 52 may include a description of a procedure type being performed, fire risk associated with the selected procedure type, and risk factors associated with the selected procedure type. In one embodiment, the system 10 may access a remote server device to access fire risk information for the selected procedure type. In another embodiment, a health care professional may register tools, surgical articles, foreseeable treatment details, etc. from a selectable list prior to the initiation of the procedure. In the illustrated example, an alcohol prep process is described as part of the procedure. The alcohol prep process includes a mandatory timer, allowing an exemplary period of sixty seconds for the alcohol to evaporate, during which no potentially inflammatory actions are to be initiated. Line item 53 is illustrated, reminding the viewer that the alcohol prep process requires activation of the timer, and a timer activation button 54 is provided, which, when pressed or activated, prompts display of a visible timer and an alarm or alert following the running of the timer when the required time period has passed. Other line items are envisioned, for example, enabling use of the described cautery device when a warning panel is acknowledged, alerting the viewer of potential risk of ignition created by use of the device before electrical power is provided to the device. The illustrated information upon primary display 21 is exemplary, and other lists, caution alerts, and precautionary measures may be implemented to prevent foreseeable fire risks.

As mentioned, one of the exemplary implementations of the surgical management system is in the context of managing and counting surgical sponges. FIG. 12 shows the surgical article 110 being a surgical sponge in which a tag 120 is disposed between opposing surfaces 112, 114 of an absorbent material body 111. Alternative surgical articles for use with the methods disclosed herein may include laparotomy pads, gauzes, implants, towels, suture needles, clips, staples, or surgical instruments, such as a scalpel or forceps. The tag 120 may be an RFID tag may be configured to convey unique identification information by transmitting an electromagnetic signal or wave to the data reader 22, or alternatively the tag may be optically read by an optical detector. The unique identification information may include a serial number or other identifier that is unique, and/or a type, size, weight, manufacturing dates, expiration date, and the like.

Referring now to FIG. 13, an exemplary method 200 of facilitating the surgical procedure is shown. The method 200 is envisioned and described herein for use with the surgical management system 10 of FIG. 1, although the method 200 may be utilized with other systems. The method 200 starts at the step 202 with the procedure (e.g., procedure information) in an unverified state. At step 204, stored procedure information, including patient information and clinical information, may be received from a database (not identified). The procedure information may be displayed on the primary display 21. A preliminary checklist is performed prior to a start to the surgical procedure to verify the procedure information (step 206). Prior to the satisfaction of the preliminary checklist, the processor 24 may prevent the counting of the surgical articles 110 to the surgical procedure with procedure information in the unverified state. The preliminary checklist may include various steps, such as confirming identity of the patient, the procedure to be performed, and identity of the medical personnel. The preliminary checklist may also include completion of other pre-procedure protocols including a series of actions to be performed before commencement of invasive aspects of the surgical procedure. The processor 24 determines the procedure information to be in a verified state based on satisfaction of the preliminary checklist, after which the processor 24 enables the counting in of the surgical articles 110 to the surgical procedure.

In certain implementations, the primary display 21 may display the tiles of grouped content, wherein at least one of the tiles of grouped content is visually deemphasized. The patient information is received based on wireless detection by the data reader 22 of a patient tag associated with a patient. The processor 24 determines the procedure information to be in a verified state based on the verifying patient information matching the stored patient information. As mentioned, the presentation of the deemphasized tile of grouped content based on the procedure information may be altered on the primary display 21 with the procedure information in the verified state. Otherwise, an alert may be provided on the primary display and/or the display interface if the verifying patient information does not match the stored patient information (step 208), in which the system may prevent activate of subsequent features of the system and provide assistance to address the discrepancy.

If the items of the preliminary checklist are determined at step 206 to have been satisfied, the method 200 advances to optional step 210, wherein a counting time-out may be enforced. During this time-out, measures may be taken to reinforce the counting process with the health care professionals that will be present during the surgical procedure. For example, professionals signed in for the procedure may have to enter an identification number or with a badge scan to the data reader 22 acknowledge compliance with the counting process. Such a time-out is shown in a variety of applications to increase compliance and serve to effectively remind professionals of required steps in a process. At step 212, the surgical procedure may commence. At step 214, sponges are counted in and counted out in accordance with the methods described herein. Throughout the surgical procedure, the count in and the count out may be displayed on the display interface 20 and/or the primary display 21 (see FIG. 6B). At step 222, the method 200 ends.

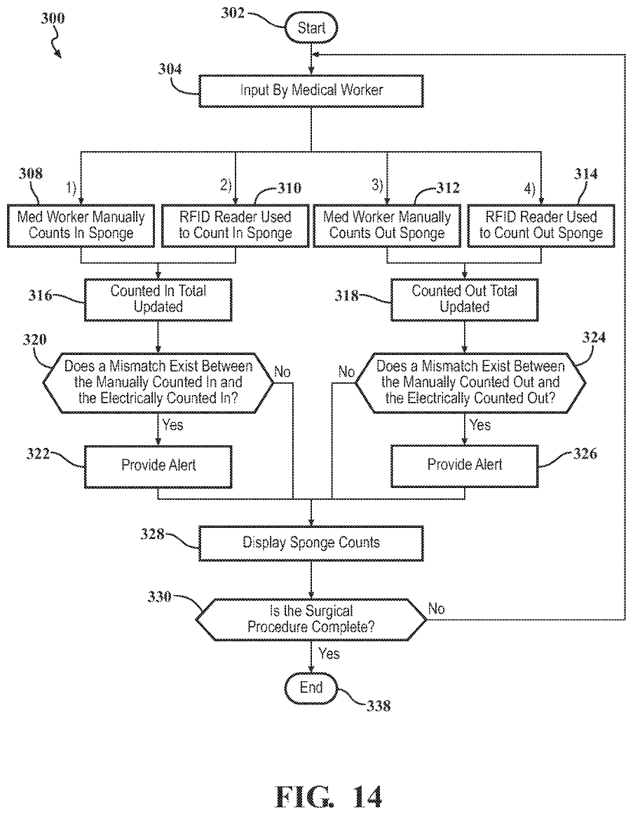

FIG. 14 is a flowchart illustrating an exemplary method 300 to count sponges utilizing manual and electronic counting steps. The method 300 is envisioned and described herein for use with the surgical sponge management system 10 of FIG. 1, although the method 300 may be utilized with other similar surgical counting systems. The method 300 starts at the step 302. At step 304, a count-related input is received by the medical worker. The count-related input may be one of a manual count in (step 308), an electronic count in (step 310), a manual count out (step 312), and an electronic count out (step 312). The manual count in and count out may be a user input to an appropriate screen the display interface 20. For example, the display interface 20 may be in a count in or count out mode, and the user may select a value corresponding to the number of sponges counted in or out, respectively. The electronic count in and count out may be performed using the data reader 24 in manners previously described.

With concurrent reference to FIG. 6B, the electronic count in and count out are updated and displayed (steps 316, 318), in particular a tally of both manually counted in sponges and electronically counted in sponges. As appreciated from the article count tile 38 of FIG. 6B, the Count (i.e., the manual count) is displayed adjacent to the Adjunct Count (i.e., the electronic count). Further, the manual count is shown as incrementally updated with each instance sponges are counted in or out the procedure. For example, FIG. 6B shows three four instances that 4×4 sponges were counted in as reflected by the struck through numbers and increasing value, and one instance where a group of 4×4 sponges were counted out as reflected by the struck through numbers and decreasing value. Since the largest numbers from the manual count is the same as those identified as counted in on the electronic count, it is deduced that the count in was accurate. A similar situation is presented for illustrative purposes with the 4×8 sponges.

At step 320, a determination is made whether a mismatch exists between the manually counted in number of sponges and the electronically counted in number of sponges. According to the manual count of FIG. 6B, one sponge of each type remains counted in. According, to the adjacent count, however, all of the sponges are identified as having been counted out. If, at step 320, a determination is made that a mismatch exists between the manually counted in number of sponges and the electronically counted in number of sponges, an alert is provided (step 322).

Regardless of whether or not there is a mismatch, the method includes displaying the sponge counts (step 328), including a tally of both manually counted out sponges and electronically counted out sponges. The method 300 continues until the surgical procedure is identified as completed (step 330), for example, with an input to the display interface 20. Subsequent to the surgical procedure, reports may be generated (e.g., accurate or resolved sponge count) for use with the administrator's workstation 23.

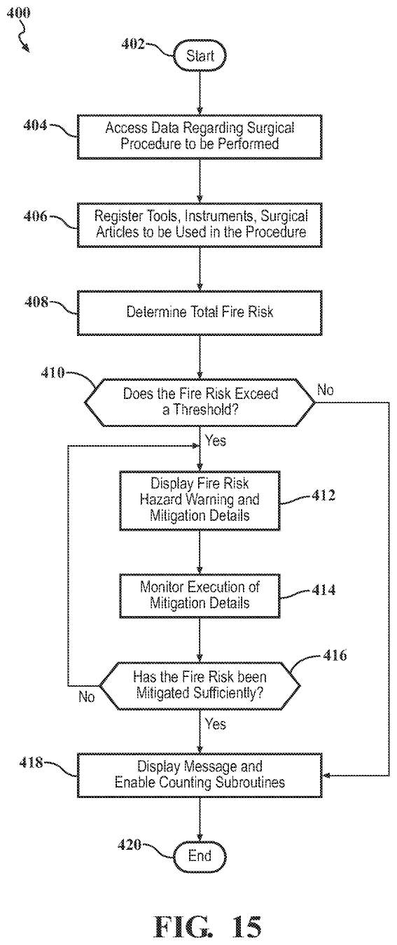

As alluded to above, many surgical procedures utilize electrocautery. Further, surgical procedures often require the use of alcohol for sterilization, and oxygen for the sedated patient. The combination of electrocautery, alcohol, and oxygen present the risk of fire by providing the heat, fuel, and oxygen, respectively, required for a fire. It is known in certain surgical procedures to assess the risk of fire. For example, certain types of surgical procedures are likely to increase the fire risk, such as those with a surgical site above the xiphoid. The surgical management system 10 of the present disclosure facilitates an intuitive and semi-automated process of assessing the fire risk. Referring to FIG. 15, a method 400 starts at the step 402. At step 404, data is accessed regarding a surgical procedure to be performed. The data may include information relative to surgical accessories (e.g., tools, instruments, materials, fluids, etc.) which are listed for use with the surgical procedure. The data may include fire risk factors for the various surgical accessories. The data may include some rules, for example, requiring a wait period after rubbing alcohol is implemented upon the patient. At step 406, a health care professional may register surgical accessories, tools, instruments, surgical articles, etc. to be used in the procedure, for example, by scanning the devices or objects with the data reader 22 or by exposing a bar code or other visual code to a camera device operated by the system 10. At step 408, the processor 24 may determine a total fire risk based upon data provided in relation to the surgical procedure to be performed and the surgical accessories scanned in by the health care professional. At step 412, the processor 24 may determine whether the total fire risk exceeds a threshold. If the total fire risk does not exceed the threshold, the method 400 advances to step 418. If the total fire risk does exceed the threshold, the method 400 advances to step 412, wherein a fire risk hazard warning is displayed, and mitigation details are provided to the health care professional. At step 414, the system 10 may monitor execution of the fire risk mitigation details. At step 416, a determination may be made whether the fire risk has been sufficiently mitigated. If the fire risk has been mitigated, the method 400 advances to the step 418. If the fire risk has not been mitigated, the method 400 returns to the step 412. At the step 418, a message is displayed stating that the fire risk has been mitigated, and a remainder of steps in the surgical procedure may be enabled, such as sponge counting methods described herein. At step 420, the method 400 ends. The method 400 is provided as an exemplary method to integrate a fire risk assessment into a surgical article counting method as disclosed herein. A number of additional or alternative method steps are envisioned, and the disclosure is not intended to be limited to the examples provided herein.

With concurrent reference to FIG. 11, the method 400 of FIG. 15 may be utilized to provide details of fire risk checklist upon primary display 21. Details of the procedure to be performed and potential fire risks associated therewith may be displayed upon primary display 21, and the health care professional may be provided with a series or list of precautionary measures selected or configured to greatly reduce the risk of a flammable substance being exposed to a potential ignition source during the procedure.

FIG. 16 is a flowchart illustrating an exemplary method 500 to determine whether a required prerequisite step has been performed in order to enable a surgical process to proceed. The method 500 is envisioned and described herein for use with the surgical sponge management system 10 of FIG. 1, although the method 500 may be utilized with other similar surgical counting systems. The method 500 starts at the step 502. At step 504, data regarding the surgical procedure to be performed is accessed. The data may include a list of surgical articles that may be utilized in the procedure. The data may include materials or treatments to be applied. In one embodiment, a surgical drape may be required for use in the procedure. At step 506, the processor 24 may determine from the data one or more necessary steps required to start the procedure or required to start a sub-procedure or portion of the procedure. At step 508, a prompt may be displayed describing the required step. In the instance of use of a surgical drape, the prompt may instruct the health care professional to deploy the surgical drape at an indicated time. At step 510, sensors, camera, user inputs, etc. may be monitored to determine performance of the required step. In the instance of required deployment of a surgical drape, the system 10 may monitor a camera device, and the health care professional, removing backing tape from adhesive upon the surgical drape, may expose a bar code only visible once the backing tape is removed to indicate to the system 10 through the camera device that the surgical drape has been deployed. At step 512, a determination is made whether the required step has been performed. If the required step has been performed, the method 500 advances to step 516. If the required step has not been performed, the method 500 advances to step 514, wherein an error message is displayed providing information about the determined missing required step, and the method 500 returns to the step 508. At the step 516, a message is displayed confirming performance of the required step, and further steps in the surgical procedure such as a sponge counting method may be enabled. At step 518, the method 500 ends. The method 500 is provided as an exemplary method to monitor occurrence of a required step in a surgical procedure and conditionally enable subsequent portions of the surgical procedure based upon determined occurrence of the required step. A number of additional or alternative method steps are envisioned, and the disclosure is not intended to be limited to the examples provided herein.

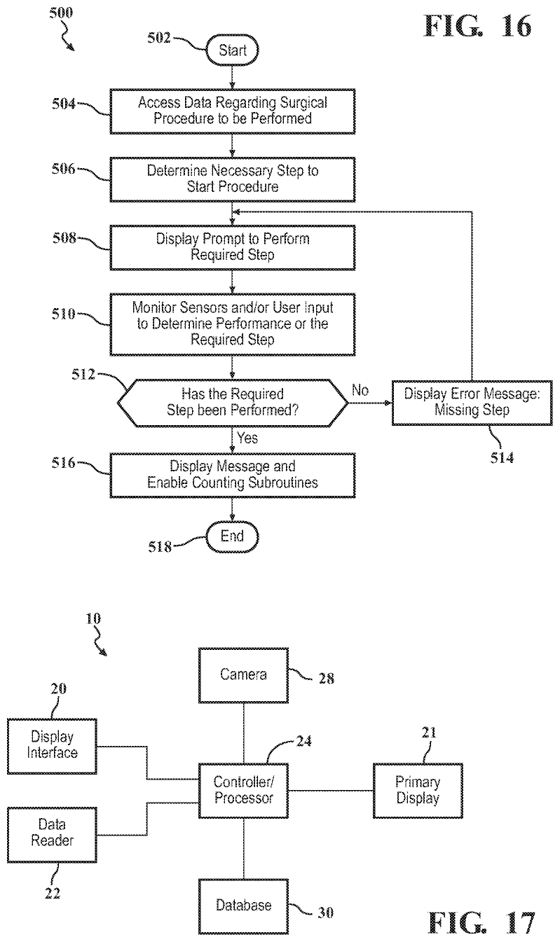

FIG. 17 schematically illustrates the system 10 configured for operating a surgical article counting method during a surgical procedure. The system 10 is illustrated including processor 24. The processor 24 may include random-access-memory (RAM) and durable memory useful to store programmable code or programmed instructions. The system 10 is further illustrated including the display interface 20, for example, including a computerized tablet useful for a health care professional to enter information such as a manually counted in sponge entry, the data reader 22, a camera 28 useful to capture images, for example, including bar code information, a sensor such as a smoke detection sensor useful to determine presence of smoke, and the primary display 21, which may be an output device or may be a touch screen device configured as an input/output device. The camera 28 may be a standalone device, for example, attached to the stand 12 of FIG. 1. In another embodiment, the camera 28 may be an embedded camera built-in to the display interface 20, for example, embodied as a commercially available tablet device.

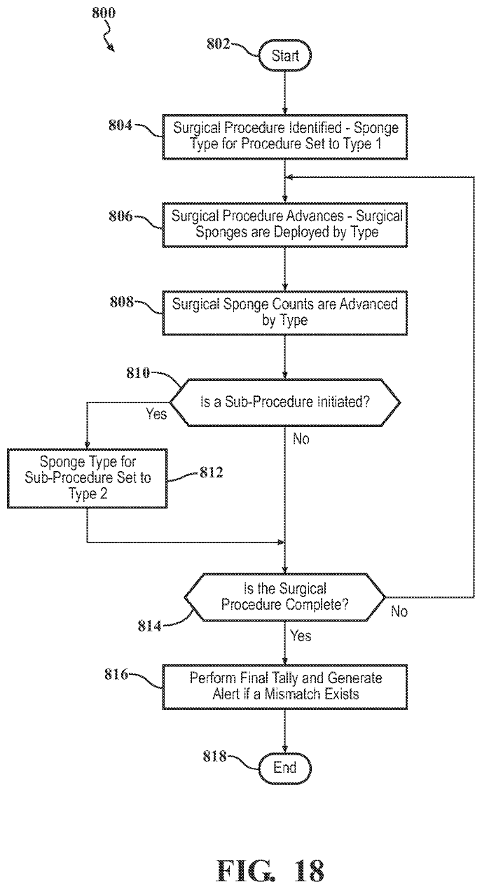

FIG. 18 is a flowchart illustrating a method 800 for initiating a sub-procedure during a primary surgical procedure and maintaining a separate sponge count during related to the sub-procedure. The method 800 is envisioned and described herein for use with the surgical sponge management system 10 of FIG. 1, although the method 800 may be utilized with other similar surgical counting systems. The method 800 starts at the step 802. At step 804, a surgical procedure is identified as a primary procedure to be performed. At least one sponge or group of sponges is identified as a type 1 sponge for use in or during the primary procedure. At step 806, the surgical procedure advances, and health care professionals deploy surgical sponges according to the needs of the surgeon. At step 808, counts in and counts out of the surgical sponges are maintained according to the methods disclosed herein according to the type of sponge being deployed. At step 810, during the surgical procedure, a determination 810 is made whether a sub-procedure, such as a procedure to treat an unexpected condition uncovered in the patient, is being initiated by the surgeon. If a sub-procedure is being initiated, the method 800 advances to the step 812, wherein a sponge type for set of sponges designated for use in the sub-procedure is set to type 2. The method 800 then advances to step 814.

The method 800 is provided as an exemplary method for implementing an unexpected sub-procedure within a surgical procedure and maintaining a separate sponge count by type between the primary procedure and the sub-procedure. A number of additional or alternative method steps are envisioned, and the disclosure is not intended to be limited to the examples provided herein.

With concurrent reference to FIG. 10, the method 800 of FIG. 18 may be utilized to provide details of concurrently tracked procedures upon primary display 21. Details of each of the identified procedures or sub-procedures to be performed may be displayed upon primary display 21. The health care professional may be provided with clear delineation of which procedure is currently active and is provided with procedure-specific details.

Certain inventive aspects of the present disclosure are made with reference to the following exemplary clauses.

Clause 1—A method of facilitating a surgical procedure with a surgical management system including a radiofrequency (RFID) reader, a processor, a user interface, and a primary display separate from the user interface, the method comprising: receiving, through a first user input to the user interface, a manual count in of a first quantity of the plurality of surgical sponges of a first sponge type being counted into the surgical procedure; displaying, on the primary display, the manual count in of the first quantity of the plurality of surgical sponges; receiving sponge identification information based on wireless detection, by the RFID reader, of an RFID tag from each of one or more of the plurality of surgical sponges to be counted into the surgical procedure, wherein the sponge identification information identifies each of the one or more of the plurality of surgical sponges to be counted into the surgical procedure as being of the first sponge type or a second sponge type; determining for each of the one or more of the plurality of surgical sponges to be counted into the surgical procedure, with the one or more processors, whether the surgical sponge associated with the RFID tag is of the first sponge type or the second sponge type; generating an electronic count in of a second quantity of the plurality of surgical sponges of the first type based on the detection of the RFID tag with the RFID reader; displaying, on the primary display, the electronic count in of the second quantity of surgical sponges of the first type; and providing, on the primary display, a first visual indication if the electronic count in of the plurality of surgical sponges of the first sponge type does not match the manual count in of the first quantity of the plurality of surgical sponges.

Clause 2—The method of clause 1, further comprising: receiving, through a second user input to the user interface, a manual count out of a third quantity of the plurality of surgical sponges of the first sponge type being counted out of the surgical procedure; displaying, on the primary display, the manual count out of the third quantity of the plurality of surgical sponges; receiving the sponge identification information based on wireless detection, by the RFID reader, of the RFID tag from each of one or more of the plurality of surgical sponges to be counted out of the surgical procedure, wherein the sponge identification information identifies each of the one or more surgical sponges to be counted out of the surgical procedure as being of the first sponge type or the second sponge type; determining for each of the one or more of the plurality of surgical sponges to be counted into the surgical procedure, with the one or more processors, whether the surgical sponge associated with the RFID tag is of the first sponge type or the second sponge type; generating an electronic count out of a fourth quantity of the plurality of surgical sponges of the first type based on the detection of the RFID tag with the RFID reader; displaying, on the primary display, the electronic count out of the fourth quantity of the plurality of surgical sponges of the first type; and providing, on the primary display, a second visual indication if the electronic count out of fourth quantity of the plurality of surgical sponges does not match the manual count out of the third quantity of the plurality of surgical sponges.

Clause 3—The method of clause 2, further comprising providing a third visual indication of how many of the plurality of surgical sponges of the first type remain counted in based upon at least one of the manual count in and the electronic count in and further based upon at least one of the manual count out and the electronic count out.

Clause 4—The method of clause 2, further comprising providing a third visual indication of how many of the plurality of surgical sponges of the first type remain counted in based upon the manual count in, the electronic count in, the manual count out, and the electronic count out.

Clause 5—The method of clause 2, wherein the first visual indication and the second visual indication are configured for communicating a potential error in counting of the plurality of surgical sponges.

Clause 6—The method of clause 2, further comprising: receiving, through a third user input to the user interface, a manual count in of a fifth quantity of the plurality of surgical sponges of the second sponge type being counted into the surgical procedure; displaying, on the primary display, the manual count in of the fifth quantity of the plurality of surgical sponges; generating an electronic count in of a sixth quantity of the plurality of surgical sponges of the second type based on the detection of the RFID tag with the RFID reader; displaying, on the primary display, the electronic count in of the sixth quantity of the plurality of surgical sponges of the second type; and providing, on the primary display, a third visual indication if the electronic count in of the sixth quantity of the plurality of surgical sponges does not match the manual count in of the fifth quantity of the plurality of surgical sponges.

Clause 7—The method of clause 6, further comprising: receiving, through a fourth user input to the user interface, a manual count out of a seventh quantity of the plurality of surgical sponges of the second sponge type being counted out of the surgical procedure; displaying, on the primary display, the manual count out of the seventh quantity of the plurality of surgical sponges; generating an electronic count out of an eighth quantity of the plurality of surgical sponges of the second type based on the detection of the RFID tag with the RFID reader; displaying, on the primary display, the electronic count out of the eighth quantity of the plurality of surgical sponges; and providing, on the primary display, a fourth visual indication if the electronic count out of the eighth quantity of the plurality of surgical sponges does not match the manual count out of the seventh quantity of the plurality of surgical sponges.

Clause 8—The method of clause 7, further comprising providing a fifth visual indication of how many of the plurality of surgical sponges remain counted in based upon the manual count in of the first quantity, the manual count in of the fifth quantity, the electronic count in of the second quantity, the electronic count in of the sixth quantity, the manual count out of the third quantity, the manual count out of the seventh quantity, the electronic count out of the fourth quantity, and the electronic count out of the eighth quantity.

Clause 9—The method of clause 7, wherein the first visual indication, the second visual indication, the third visual indication, and the fourth visual indication are configured for communicating a potential error in counting of the plurality of surgical sponges.

Clause 10—The method of clause 7, wherein a portion of the plurality of sponges being of the first type are the portion of the plurality of sponges manually counted in by a first medical user, wherein a portion of the plurality of sponges being of the second type are the portion of the plurality of sponges manually counted in by a second medical user.

Clause 11—The method of clause 7, wherein a portion of the plurality of sponges being of the first type are the portion of the plurality of sponges manually counted in by a one or more medical workers of a first working shift which ends prior to a second working shift starting, wherein a portion of the plurality of sponges being of the second type are the portion of the plurality of sponges manually counted in by one or more medical workers of the second working shift.

Clause 12—The method of clause 7, wherein a portion of the plurality of sponges being of the first type are the portion of the plurality of sponges manually counted in during a first portion of the surgical procedure, wherein a portion of the plurality of sponges being of the second type are the portion of the plurality of sponges manually counted in during a second portion of the surgical procedure.

Clause 13—The method of clause 12, wherein the first portion of the surgical procedure includes a primary portion of the surgical procedure, wherein the second portion of the surgical procedure includes an ancillary portion of the surgical procedure.

Clause 14—The method of clause 12 wherein the first portion of the surgical procedure includes a planned portion of the surgical procedure, wherein the second portion of the surgical procedure includes an emergent portion of the surgical procedure necessitated by new information discovered during the planned portion.

Clause 15—The method of clause 12, wherein the first portion of the surgical procedure includes a portion associated with a first surgeon, wherein the second portion of the surgical procedure includes a portion associated with a second surgeon.

Clause 16—The method of clause 12, wherein the first portion of the surgical procedure includes a portion of the surgical procedure dealing with a quarantined region of a patient, wherein the second portion of the surgical procedure includes a portion of the surgical procedure dealing with a region of the patient outside of the quarantined region.

Clause 17—The method of clause 1, wherein the first visual indication is configured for communicating a potential error in counting of the plurality of surgical sponges.

Clause 18—The method of clause 1, further comprising: receiving, through a second user input to the user interface, a manual count in of a third quantity of the plurality of surgical sponges of the second sponge type being counted into the surgical procedure; displaying, on the primary display, the manual count in of the third quantity of the plurality of surgical sponges; generating an electronic count in of a fourth quantity of the plurality of surgical sponges of the second type based on the detection of the RFID tag with the RFID reader; displaying, on the primary display, the electronic count in of the fourth quantity of the plurality of surgical sponges of the second type; and providing, on the primary display, a second visual indication if the electronic count in of the fourth quantity of the plurality of surgical sponges does not match the manual count in of the third quantity of the plurality of surgical sponges.

Clause 19—The method of clause 1, further comprising: prior to utilizing the plurality of surgical sponges, temporarily disabling the manual count in and receiving sponge identification information of the RFID tag; monitoring successful entry of a first form of patient identification verification; monitoring successful entry of a second form of patient identification verification; and upon monitoring the successful entry of the first form of patient identification verification and monitoring the successful entry of the second form of patient identification, selectively enabling receiving the manual count in and receiving sponge identification information of the RFID tag.

Clause 20—The method of clause 1, further comprising: prior to utilizing the plurality of surgical sponges, temporarily disabling the manual count in and receiving sponge identification information of the RFID tag; monitoring successful entry of a form of patient identification verification; monitoring successful entry of a procedure to be performed verification; and upon monitoring the successful entry of the form of patient identification verification and monitoring the successful entry of the procedure to be performed verification, selectively enabling receiving the manual count in and receiving sponge identification information of the RFID tag.

Clause 21—The method of clause 1, further comprising: prior to utilizing the plurality of surgical sponges, temporarily disabling the manual count in and receiving sponge identification information of the RFID tag; monitoring successful entry of a left-side right-side procedure site verification; and upon monitoring the successful entry of the left-side right-side procedure site verification, selectively enabling receiving the manual count in and receiving sponge identification information of the RFID tag.

Clause 22—The method of clause 1, further comprising implementing a procedure time-out configured for emphasizing counting of the plurality of surgical sponges.

Clause 23—The method of clause 1, further comprising monitoring a sensor providing details of the surgical procedure; and selectively enabling portions of the surgical procedure based upon the details from the sensor.

Clause 24—The method of clause 23, wherein monitoring the sensor providing the details includes monitoring a camera device capturing images of the surgical procedure.

Clause 25—The method of clause 24, wherein monitoring the camera device capturing images of the surgical procedure includes monitoring exposure of a QR code configured to be revealed when a piece of surgical equipment is utilized.

Clause 26—The method of clause 24, wherein monitoring the camera device capturing images of the surgical procedure includes monitoring deployment of a flammable substance; further comprising evaluating a fire risk posed by the deployment of the flammable substance, wherein selectively enabling portions of the surgical procedure includes providing, on the primary display, a second visual indication of a fire hazard if the fire risk exceeds a threshold.

Clause 27—The method of clause 26, further comprising: identifying a time-sensitive fire risk; and initiating a timer based upon the time-sensitive fire risk.

Clause 28—The method of clause 23, wherein monitoring the sensor providing the details includes monitoring activation of a surgical tool to be utilized in the surgical procedure.

Clause 29—The method of clause 28, wherein monitoring activation of the surgical tool includes evaluating a fire risk posed by the surgical tool in light of stored parameters of the surgical procedure.

Clause 30—The method of clause 1, further comprising providing a selectable user input which causes the primary display to display an evaluation of how many of the plurality of surgical sponges are currently counted in.

Clause 31—The method of clause 1, further comprising displaying a color-coded summary of the plurality of surgical sponges, with a first color being utilized to provide details regarding a first portion of the plurality of sponges of the of the first type and with a second color being utilized to provide details regarding a second portion of the plurality of sponges of the second type.

Clause 32—A method of facilitating a surgical procedure with a surgical management system including a radiofrequency (RFID) reader, a processor, a user interface, and a primary display separate from the user interface, the method comprising: utilizing a plurality of surgical sponges during the surgical procedure including: a first portion of the plurality of surgical sponges being of a first type and being counted in by a first medical user working upon a first shift occurring relatively earlier in time; and a second portion of the plurality of surgical sponges being of a second type and being counted in by a second medical user working upon a second shift occurring relatively later in time; receiving, through a first user input to the user interface, a manual count in of a first quantity of the plurality of surgical sponges of a first sponge type being counted into the surgical procedure; displaying, on the primary display, the manual count in of the first quantity of the plurality of surgical sponges; receiving sponge identification information based on wireless detection, by the RFID reader, of an RFID tag from each of one or more of the plurality of surgical sponges to be counted into the surgical procedure, wherein the sponge identification information identifies each of the one or more of the plurality of surgical sponges to be counted into the surgical procedure as being of the first sponge type or a second sponge type; determining for each of the one or more of the plurality of surgical sponges to be counted into the surgical procedure, with the one or more processors, whether the surgical sponge associated with the RFID tag is of the first sponge type or the second sponge type; generating an electronic count in of a second quantity of the plurality of surgical sponges of the first type based on the detection of the RFID tag with the RFID reader; displaying, on the primary display, the electronic count in of the second quantity of surgical sponges of the first type; providing, on the primary display, a first visual indication if the electronic count in of the plurality of surgical sponges of the first sponge type does not match the manual count in of the first quantity of the plurality of surgical sponges; receiving, through a second user input to the user interface, a manual count in of a third quantity of the plurality of surgical sponges of the second sponge type being counted into the surgical procedure; displaying, on the primary display, the manual count in of the third quantity of the plurality of surgical sponges; generating an electronic count in of a fourth quantity of the plurality of surgical sponges of the second type based on the detection of the RFID tag with the RFID reader; displaying, on the primary display, the electronic count in of the fourth quantity of the plurality of surgical sponges of the second type; providing, on the primary display, a third visual indication if the electronic count in of the sixth quantity of the plurality of surgical sponges does not match the manual count in of the fifth quantity of the plurality of surgical sponges; and displaying, on the primary display: details of the first portion of the plurality of sponges; and details of the second portion of the plurality of sponges in a distinct manner from the details of the first portion of the plurality of sponges

Clause 33—The method of clause 32, wherein displaying the details of the second portion of the plurality of sponges in the distinct manner includes displaying the details of the first portion in a first color and displaying details of the second portion in a second color.

Clause 34—The method of clause 32, wherein displaying the details of the second portion of the plurality of sponges in the distinct manner includes displaying the details of the first portion upon a first portion of the primary display and displaying details of the second portion a second portion of the primary display distal from the first portion of the primary display.

Clause 35—A method of facilitating a surgical procedure with a surgical management system including a radiofrequency (RFID) reader, a processor, a user interface, and a primary display separate from the user interface, the method comprising: utilizing a plurality of surgical sponges during the surgical procedure including: a first portion of the plurality of surgical sponges being of a first type and being counted in during a first sub-procedure of the surgical procedure; and a second portion of the plurality of surgical sponges being of a second type and being counted in during a second sub-procedure of the surgical procedure; receiving, through a first user input to the user interface, a manual count in of a first quantity of the plurality of surgical sponges of a first sponge type being counted into the surgical procedure; displaying, on the primary display, the manual count in of the first quantity of the plurality of surgical sponges; receiving sponge identification information based on wireless detection, by the RFID reader, of an RFID tag from each of one or more of the plurality of surgical sponges to be counted into the surgical procedure, wherein the sponge identification information identifies each of the one or more of the plurality of surgical sponges to be counted into the surgical procedure as being of the first sponge type or a second sponge type; determining for each of the one or more of the plurality of surgical sponges to be counted into the surgical procedure, with the one or more processors, whether the surgical sponge associated with the RFID tag is of the first sponge type or the second sponge type; generating an electronic count in of a second quantity of the plurality of surgical sponges of the first type based on the detection of the RFID tag with the RFID reader; displaying, on the primary display, the electronic count in of the second quantity of surgical sponges of the first type; providing, on the primary display, a first visual indication if the electronic count in of the plurality of surgical sponges of the first sponge type does not match the manual count in of the first quantity of the plurality of surgical sponges; receiving, through a second user input to the user interface, a manual count in of a third quantity of the plurality of surgical sponges of the second sponge type being counted into the surgical procedure; displaying, on the primary display, the manual count in of the third quantity of the plurality of surgical sponges; generating an electronic count in of a fourth quantity of the plurality of surgical sponges of the second type based on the detection of the RFID tag with the RFID reader; displaying, on the primary display, the electronic count in of the fourth quantity of the plurality of surgical sponges of the second type; providing, on the primary display, a third visual indication if the electronic count in of the sixth quantity of the plurality of surgical sponges does not match the manual count in of the fifth quantity of the plurality of surgical sponges; and displaying, on the primary display: details of the first portion of the plurality of sponges; and details of the second portion of the plurality of sponges in a distinct manner from the details of the first portion of the plurality of sponges.

Clause 36—The method of clause 35, wherein displaying the details of the second portion of the plurality of sponges in the distinct manner includes displaying the details of the first portion in a first color and displaying details of the second portion in a second color.

Clause 37—The method of clause 35, wherein displaying the details of the second portion of the plurality of sponges in the distinct manner includes displaying the details of the first portion upon a first portion of the primary display and displaying details of the second portion a second portion of the primary display distal from the first portion of the primary display.

The foregoing disclosure is not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

1. A method of facilitating a surgical procedure with a surgical management system including a data reader, one or more processors, a display interface, and a primary display separate from the display interface, the method comprising:
receiving, through a first user input to the display interface, a manual count in of surgical articles being counted in to the surgical procedure;
displaying, on the primary display, a manual count of the surgical articles based on the manual count in;
receiving, at the one or more processors, identification information based on wireless detection, with the data reader, an identification tag from one or more of the surgical articles being counted in to the surgical procedure;
determining, with the one or more processors, an electronic count in of the surgical articles based on the identification information;
displaying, on the primary display, the manual count and the electronic count in; and
providing, on the primary display, a visual indication if the manual count of the surgical articles does not match the electronic count in of the surgical articles.

2. The method of claim 1, further comprising:
receiving, through a second user input to the display interface, a manual count out of one or more of the of the surgical articles being counted out of the surgical procedure, wherein the one or more surgical articles were previously counted in to the surgical procedure;
updating, with the one or more processors, the manual count of the surgical articles based on the manual count in and the manual count out;
displaying, on the primary display, the updated manual count;
receiving the identification information based on wireless detection, with the data reader, of the identification tag from one or more of the surgical articles to be counted out of the surgical procedure;
determining, with the one or more processors, an electronic count out the surgical articles based on the identification information of the one or more articles to be counted out;
displaying, on the primary display, the manual count, the electronic count in, the electronic count out; and
providing, on the primary display, the visual indication if the manual count of the surgical articles does not match the electronic count out of the surgical articles.

3. The method of claim 2, further comprising:
determining, with the one or more processors, an electronic count of articles remaining as counted in as a difference between the electronic count in and the electronic count out; and
displaying, on the primary display, the electronic count of articles remaining as counted in; and
providing, on the primary display, the visual indication if the manual count of the surgical articles does not match the electronic count of articles remaining as counted in.

4. The method of claim 1, wherein each entry of the manual count in and the manual count out prior to an updated entry remain displayed on the primary display and indicated as a previous entry, and, optionally, wherein the previous entry is struckthrough.

5. The method of claim 1, wherein the manual count in is inputted by a first medical staff member, the method further comprising:
receiving, on the display interface or via the data reader, an identification input indicative of a change in staffing;
receiving, through a third user input to the display interface by a second medical staff member, a second manual count in of additional surgical articles being counted in to the surgical procedure;
updating, with the one or more processors, the manual count of the surgical articles based on the manual count in and the second manual count in; and
displaying, on the primary display, the updated manual count including the manual count in and the second manual count in,
wherein the manual count in and the second manual count are displayed to identify as being inputted by different medical staff members.

6. The method of claim 5, wherein the identification as being entered by different medical staff include text of different color.

7. The method of claim 5, wherein the identification input is an optical barcode associated with each of the first and second medical staff members.

8. The method of claim 1, further comprising:
associating, on the display interface, the manual count with a first portion of the surgical procedure;
generating, on the display interface, indicia associated with a second portion of the surgical procedure; and
receiving, through another input on the display interface, another manual count in of surgical articles being counted in to the second portion of the surgical procedure; and
displaying, on the primary display, another manual count of the surgical articles in a display screen separate from a display screen associated with the manual count associated with the first portion of the surgical procedure.

9. The method of claim 8, further comprising receiving a toggle input to selectively toggle between article counts associated with the first portion of the surgical procedure and the second portion of the surgical procedure.

10. The method of claim 8, wherein the second portion of the surgical procedure is based on an emergent portion of the surgical procedure necessitated by information discovered during the first portion.

11. The method of claim 8, wherein the first portion of the surgical procedure is associated with a first surgeon, and wherein the second portion of the surgical procedure is associated with a second surgeon.

12. The method of claim 1, further comprising:
prior to initiating surgical aspects of the surgical procedure, preventing, with the one or more processors, the data reader from counting in of surgical articles to the surgical procedure;
receiving, on the display interface, inputs configured to satisfy a pre-procedure protocol; and
enabling, with the one or more processors, the data reader for the counting in of the surgical articles to the surgical procedure based on satisfaction of the pre-procedure protocol.

13. The method of claim 12, wherein the pre-procedure protocol is selected from the group consisting of verification of patient information, verification of procedure information, and fire risk assessment.

14. The method of claim 12, further comprising
displaying, on the primary display, tiles of grouped content, wherein at least one of the tiles of grouped content is visually deemphasized; and
altering, on the primary display, presentation of the deemphasized tile of grouped content based on satisfaction of the pre-procedure protocol.

15. The method of claim 1, wherein the surgical articles are one of surgical sponges and surgical instruments, and optionally, wherein the data reader is a radiofrequency identification (RFID) reader and the identification tag is an RFID tag.

16. A method of facilitating a surgical procedure with a surgical management system including a data reader, one or more processors, a display interface, and a primary display separate from the display interface, the method comprising:
preventing, with the one or more processors, counting in of surgical articles to the surgical procedure with procedure information in an unverified state;
displaying, on the primary display, tiles of grouped content, wherein at least one of the tiles of grouped content is visually deemphasized;
receiving, from a database, stored procedure information including patient information and clinical information;
displaying, on the primary display, the patient information;
receiving patient information based on wireless detection by the data reader of a patient tag associated with a patient;
determining, with the one or more processors, the procedure information to be in a verified state based on the patient information matching the stored patient information;
altering, on the primary display, presentation of the deemphasized tile of grouped content based on the procedure information being in the verified state; and
enabling, with the one or more processors, the counting in of the surgical articles to the surgical procedure based on the procedure information being in the verified state.

17. The method of claim 16, further comprising providing an alert on the primary display and/or the display interface if the patient information does not match the stored patient information.

18. (canceled)

19. (canceled)

20. A method of facilitating a surgical procedure with a surgical management system including a data reader, one or more processors, a display interface, and a primary display separate from the display interface, the method comprising:
preventing, with the one or more processors, counting in of surgical articles to the surgical procedure with procedure information in an unverified state;
displaying, on the primary display, a pre-procedure protocol including a series of actions to be performed before commencement of invasive aspects of the surgical procedure;
receiving, from a database, stored procedure information including patient information and clinical information;
displaying, on the primary display, the patient information and the clinical information;
receiving, on the display interface, inputs configured to satisfy each of the series of actions of the pre-procedure protocol;
altering, on the primary display, indicia corresponding to each of the series of actions as each of the inputs is determined to have been performed, by the one or more processors, to satisfy a respective one of the series of actions;
determining, with the one or more processors, the procedure information to be in a verified state based all of the series of actions of the pre-procedure protocol being satisfied; and
enabling, with the one or more processors, the counting in of the surgical articles to the surgical procedure based on the procedure information being in the verified state.

21. The method of claim 20, wherein the pre-procedural protocol is displayed in a tile of grouped content that is visually deemphasized with the procedure information in the unverified state.

22. The method of claim 21, wherein one of the series of actions includes confirming surgical personnel, the method further comprising:
receiving personnel information based on wireless detection by the data reader of a personnel tag associated with the surgical personnel; and
determining, with the one or more processors, whether the personnel information matches the clinical information of the stored procedure information.

23-36. (canceled)
